(19) United States
(12) Patent Application Publication
(10) Pub. No.: US 2024/0142494 A1
(43) Pub. Date: May 2, 2024

(54) PEN PROBE WITH NON-PRECIOUS METAL WIRE FOR USE IN PRECIOUS METAL TESTING APPARATUS

(71) Applicant: Jarrett Schaffer, Allentown, PA (US)

(72) Inventor: Jarrett Schaffer, Allentown, PA (US)

(21) Appl. No.: 18/207,421

(22) Filed: Jun. 8, 2023

Related U.S. Application Data
(60) Provisional application No. 63/381,112, filed on Oct. 26, 2022.

(51) Int. Cl. G01R 1/067; G01N 33/2045

(52) U.S. Cl. CPC G01R 1/067; G01N 33/2045

(57) ABSTRACT

A digital precious metal testing apparatus utilizes a test pen probe having therewithin a titanium wire that generates a galvanic voltage when an electrical circuit is completed with the object being tested between the pen probe and the meter test pad. A calibration system is provided to enhance the accuracy of the testing apparatus by comparing a test reading from a known test specimen with a corresponding theoretical reading for that specimen, and generating a recalibration curve from which all subsequent readings will be compared to determine the content of precious or non-precious metal being tested. The titanium wire test pen probe generates a greater galvanic charge compared to conventional platinum wire pen probes, which enables the test pen to be used to distinguish grades of non-precious metals, such as distinguishing grade 304 stainless steel from grade 316 stainless steel, for use in the recycling industry.

### CROSS-REFERENCE TO RELATED APPLICATIONS

This application claims domestic priority on U.S. Provisional Patent Application Ser. No. 63/381,112, filed on Jun. 10, 2022, the content of which is incorporated herein by reference.

### FIELD OF THE INVENTION

The present invention relates generally to an apparatus for testing the purity of precious metals, including gold, silver and platinum, and, more particularly, to a testing pen probe that utilizes a non-precious metal wire to measure the voltage differential in the testing apparatus.

### BACKGROUND OF THE INVENTION

A precious metal testing apparatus is shown in U.S. Pat. No. 5,888,362, issued to Lloyd V. Fegan, Jr., on Mar. 30, 1999. This testing apparatus is a portable device that can provide accurate analysis of the quality of the precious metal being tested by utilizing a hand-held probe having an electrode embedded in an electrolyte contained within a reservoir formed in the probe. The testing apparatus generates a galvanic current through the metal being tested from a battery, the strength of the current being proportionate to the quality of the precious metal being tested. In the Fegan patent, a meter circuit measures the extent of galvanic action of dissimilar metals in the presence of an electrolyte, one of the metals being the sample being tested for quality. Thus, the invention is useful for testing the metal content of coins, art objects, jewelry, and the like, by reason that the probe can simply be touched against the object being tested to provide a reading representing the quality of the precious metal in the object.

The hand-held probe in the aforementioned precious metal testing apparatus is typically in the form of a pen having a fibrous tip from which a small amount of electrolyte is deposited onto the object being tested. The meter attached to the probe continuously measures the strength of the galvanic current and compares the result with a known point of reference for the type of precious metal being tested; whereby the percentage of precious metal within the object being tested will be know. This measurement process by the meter and pen is completed within a few hundredths of a second, thus providing an efficient manner in which the quality of precious metal can be determined. However, even though the measurement process is fast, the strengths of the galvanic reaction when reacted with gold, silver or platinum are very weak.

Accordingly, slight variations in system parameters are significant enough to reduce the overall accuracy of the meter, and can render the testing apparatus essentially useless. In addition to the electronic and mechanical variations that affect the accuracy of the testing apparatus, variations in operating environment, such as temperature and humidity, also affect the accuracy of the meter. One skilled in the art will recognize that with any galvanic reaction occurring from dissimilar metals and an electrolyte, one metal is sacrificed to the other across the electrolyte. As the electrolyte and the sacrificial metal are consumed, the galvanic strength varies. Furthermore, the accuracy of the testing apparatus is impacted negatively over time because the strength of the galvanic reaction decreases with each and every specimen measured. The exhaustion of materials within the pen that are responsible for generating the galvanic strength can be mitigated by replacing an improperly functioning pen probe with a new pen probe. When the pen is replaced, however, the replacement pen will have subtle differences in composition from the pen that has been replaced, which again provides a variation that impacts the accuracy of the testing apparatus.

Calibrating the Fegan precious metal testing apparatus was possible by removing the housing and utilizing a screwdriver or similar instrument to adjust one of the variable resistors in the Fegan circuitry. Testing against a known purity of gold would provide a calibrated reading that would often need further calibration, again requiring the removal of the meter housing and further adjustment of one of the variable resistors. This process was repeated until the Fegan precious metal testing meter was showing the proper results from the known purity sample being tested for the purposes of calibrating the meter. Thus, calibrating the Fegan meter was a complex and time consuming process. Accordingly, accuracy in testing precious metals with the Fegan meter was not consistent as changes in the electrolyte and other environmental factors would deteriorate accurate test results.

A solution for solving the accuracy issues of the aforementioned testing apparatus was established as defined in U.S. Pat. No. 9,002,659, granted on Apr. 7, 2015, to Jarrett Schaffer, et al., which is directed to a calibration method to be used upon initiation of the testing process and after repetitive usage. The calibration process uses a known Karat quality of the precious metal to be tested and allow the reading for the galvanic reaction of the known calibration specimen determine the calibration chart to be selected for subsequent readings. A potential problem with this precious metal testing process is that the galvanic reaction is measured in millivolts, and variations in environmental parameters, or a contamination of the test specimen can result in significant variation in the test galvanic reaction.

Another problem with the '659 testing apparatus is that the wire within the pen probe is made from platinum or is a wire that has a heavy platinum coating. Such precious metal wires are conventional and generates a galvanic reaction, but the difference in the galvanic reaction (i.e., delta) for the higher quality test specimens being tested, such as 22K and 24K gold, which leads to a lack of conviction that the reading is correct. Gold sales outside of the United States typically involve these higher quality precious metals. Accordingly, a greater precision in the testing process is highly desired. Using platinum to be the sacrificial wire within the pen probe results in a significant cost with respect to manufacturing. Therefore, reduction is manufacturing costs would also be a desirable trait.

Furthermore, the stability of the precious metal testing apparatus is highly susceptible to changes in atmospheric conditions and to any contamination of the specimen being evaluated. The use of the platinum or platinum coated wire generating the galvanic reaction generates a small millivolt reaction that increases slightly as the quality of the metal being tested increases, i.e., from 22K to 24K. With the platinum or platinum-coated wires, the increase from 18K, which is the standard specimen used to zero the calibration, to 22K a delta of about 17 millivolts is generated. For a four carat increase that delta averages about 4 mv per Karat. Thus, changes in humidity or contamination of the specimen being tested can significantly reduce the galvanic reaction and make the detection of 22K and 24K gold specimens more difficult and less reliable.

Accordingly, it would be desirable to provide a more stable apparatus for testing precious metals.

### SUMMARY OF THE INVENTION

It is an object of this invention to overcome the disadvantages of the prior art by providing a precious metal testing apparatus that utilizes non-precious metal wire to measure the voltage differential in the testing apparatus.

It is an advantage of this invention that the pen probe establishes a galvanic reaction through the use of a non-precious metal wire within the test pen apparatus that generates a galvanic voltage in direct proportion to the percentage of precious metal content within an object being tested.

It is another object of this invention to provide a test apparatus that can be utilized to distinguish certain grades of non-precious metal samples.

It is a feature of this invention to provide a wire within the test pen apparatus that will establish a more reliable galvanic reaction that the customary platinum wire test pen apparatus.

It is another feature of this invention that the use of a titanium wire within the test pen apparatus generates a galvanic charge that is multiple times the galvanic charge obtained with a customary platinum wire.

It is another advantage that the use of a titanium wire within the test pen probe is less susceptible to variations induces by changes in atmospheric conditions and contaminations of the precious metal specimen being tested.

It is another advantage of this invention that the use of a titanium wire within the test pen apparatus generates multiple times the galvanic charge compared to the customary platinum wire without increasing the variability in the galvanic charge generated for a particular sample being tested.

It is still another advantage of this invention that the reliability of a titanium wire test pen apparatus is greater than the customary platinum wire.

It is yet another advantage of this invention that the cost of manufacturing titanium wires for use in the test pen apparatus is significantly less than the conventional platinum wires.

It is still another object of this invention to provide a testing apparatus that is operable to distinguish certain grades of non-precious metal samples.

It is still another feature of this invention that the titanium wire test pen apparatus can be used to distinguish grade 304 stainless steel from grade 316 stainless steel.

It is yet another advantage of this invention that recycling companies can utilize an inexpensive metal testing apparatus to replace the use of expensive XRF guns that are currently being used to distinguish between grade 304 and grade 316 stainless steel.

It is yet another object of this invention to provide a testing apparatus for determining the content of precious metal within an object being tested, that is durable in construction, inexpensive of manufacture, carefree of maintenance, facile in assemblage, and simple and effective in use.

It is a yet another object of this invention to provide a test pen utilizing a titanium wire for a precious metal testing apparatus that is simple and effective in use and operation to increase the accuracy of the testing apparatus.

These and other objects, features and advantages are accomplished according to the instant invention by providing a digital precious metal testing apparatus, which utilizes a test pen probe having therewithin a titanium wire that generates a galvanic voltage when an electrical circuit is completed with the object being tested between the pen probe and the meter test pad. A calibration system is provided to enhance the accuracy of the testing apparatus by comparing a test reading from a known test specimen with a corresponding theoretical reading for that specimen, and generating a recalibration curve from which all subsequent readings will be compared to determine the content of precious or non-precious metal being tested. The titanium wire test pen probe generates a greater galvanic charge compared to conventional platinum wire pen probes, which enables the test pen to be used to distinguish grades of non-precious metals, such as distinguishing grade 304 stainless steel from grade 316 stainless steel, for use in the recycling industry.

### BRIEF DESCRIPTION OF THE DRAWINGS

The advantages of this invention will be apparent upon consideration of the following detailed disclosure of the invention, especially when taken in conjunction with the accompanying drawings wherein:

FIG. 1 is a schematic diagram of a testing apparatus incorporating the principles of the instant invention and including a pen probe electrically coupled to a meter to measure the galvanic current generated by the testing apparatus and provide an output indicating the purity of the precious metal being tested;

FIG. 2 is a vertical cross-sectional view of a pen probe forming a part of the testing apparatus utilizing a pair of thin wires within the casing;

FIG. 3 is a schematic view of the electronic circuit forming the apparatus measuring the galvanic current generated through the testing procedure;

FIG. 4 is a graphic representation of the calibration process incorporating the principles of the instant invention;

FIG. 5 is a graphic representation of the corrected curve following the calibration procedure;

FIGS. 6-9 are graphic representations of the calibration procedure;

FIG. 10 is a logic flow diagram of the initial start-up sequence for the meter;

FIG. 11 is a logic flow diagram for the recalibration procedure; and

FIG. 12 is a vertical cross-sectional view of an alternative configuration of a pen probe utilizing a single wire within the casing, incorporating the principles of the instant invention.

### DETAILED DESCRIPTION OF THE PREFERRED EMBODIMENT

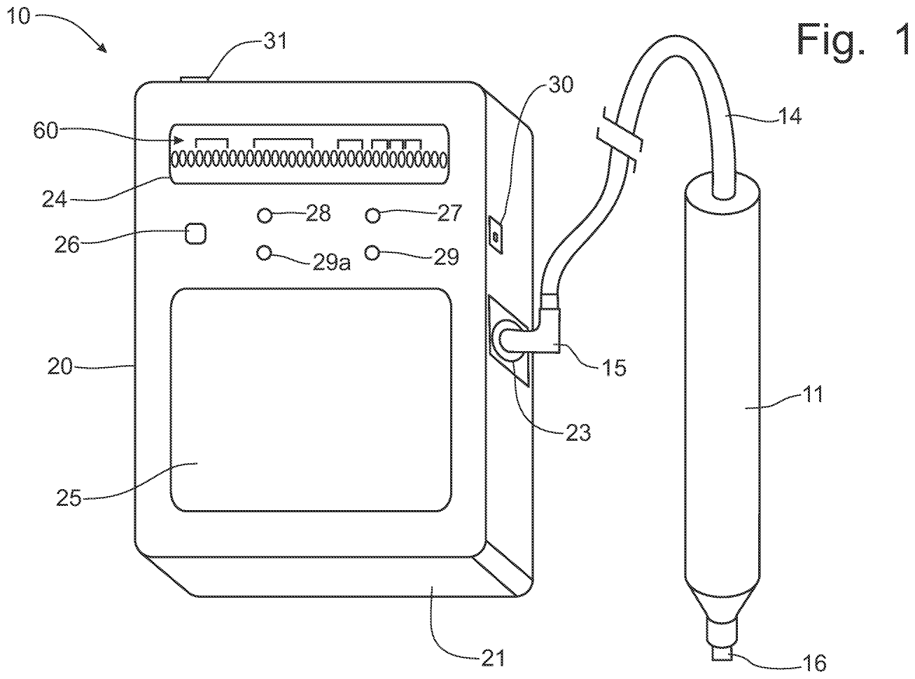

Referring now to FIGS. 1-3, a testing apparatus for analyzing the quality of the precious metal within an object being tested can best be seen. This testing apparatus 10 incorporates the principles of the instant invention by utilizing a recalibration system to enhance the accuracy of the continued use of the testing apparatus 10. The principle for the testing apparatus 10 is described in greater detail in the aforementioned U.S. Pat. No. 5,888,362 and in U.S. Pat. No. 9,002,659, the contents of which are incorporated herein by reference.

In general, the testing apparatus 10 utilizing a titanium wire 18 can be used to analyze the content or quality of gold or other precious metal, but also the quality or grade of stainless steel and other materials, and is based on developing and measuring an electromotive force (EMF) due to electrical conduction between two dissimilar metals or metal alloys, namely an electrode and the object to be analyzed. The galvanic voltage results from the difference in availability of electrons in the different metals or alloys, and provides a net current when the metals or alloys are coupled through an electrolyte. The galvanic voltage generated by the dissimilar metals or metal alloys is generated using the touch probe 11 of the invention, and is measured by subtracting the galvanic EMF from that of a battery or similar reference voltage level at a constant reverse polarity voltage. The galvanic voltage generated is directly related to the proportion of the gold or other precious metal content of the object being tested and, thus, provides a means by which the purity of the sample can be determined.

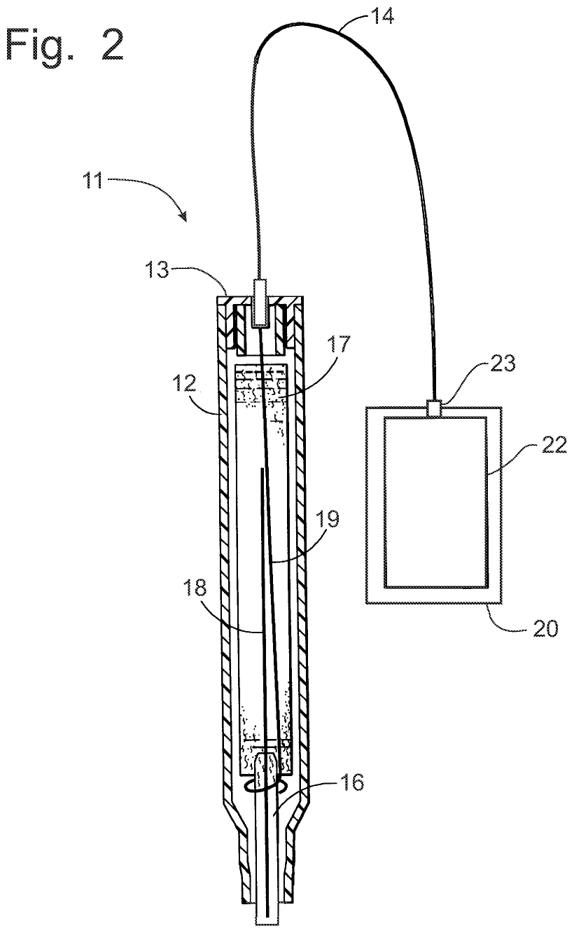

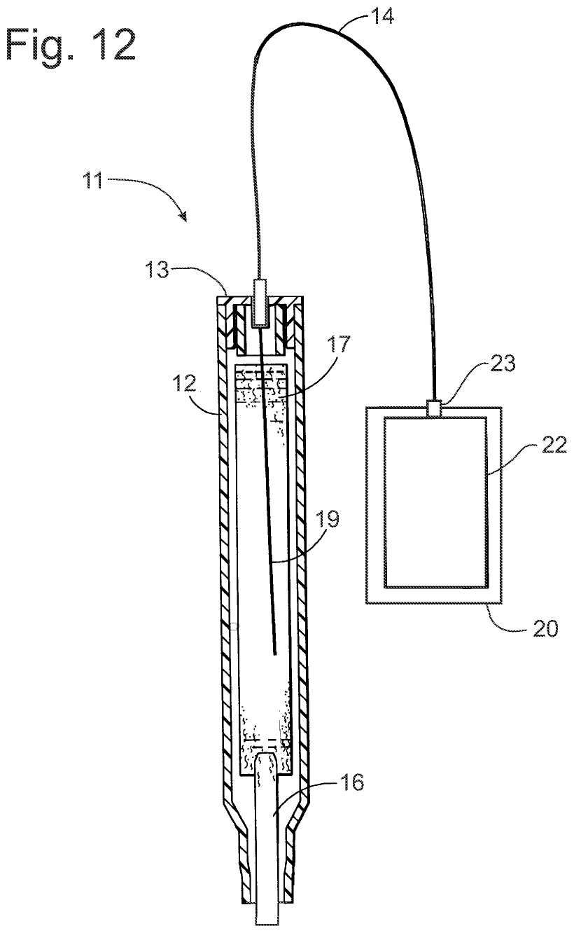

An exemplary embodiment of the pen probe 11 forming part of the testing apparatus 10 is shown in FIG. 2, which is a cross-sectional view of the exemplary hand-held pen probe 11. The pen probe 11 is preferably formed with a generally cylindrical body 12, which can be made of plastic or other substantially electrically non-conductive material. A top cap 13 is coupled to an electrical wire 14 fitted with a jack 15 to facilitate a detachable electrical connection of the pen probe 11 to the circuitry 22 within the meter 20. The end of pen probe 11, which is placed into contact with the object to be tested, is formed as a fiber tip 16 that is in communication with a reservoir 17 containing a supply of electrolyte, such as a saturated solution of ammonium chloride, or other solutions as is known for conventional galvanic testing techniques. The fiber tip 16 can be provided with a thin wire 18, which is embedded into the fiber tip 16 and extends into the reservoir 17. In this configuration, a second thin connecting wire 19 can also be utilized to connect to the jack 15 and the wire 14 at the top cap 13 of the pen probe 11. The preferred construction, however, would be to provide a single thin wire 19 connected to the jack 15 and extend into the reservoir of electrolyte as is depicted in FIG. 12. Commercial embodiments of this configuration have used a 40 mm wire.

In the aforementioned prior art patents, these two thin wires 18, 19, or alternatively, the single thin wire configuration, were all formed from platinum, or at least used a heavy platinum coating. The end result, as expressed above, was a lack of confidence in the testing of higher Karat gold. Applicant has replaced the platinum wire with a titanium wire, which has provided surprisingly superior test results. The testing process with the pen probe 11 being manufactured with a 40 mm titanium wire is the same as expressed above. A known specimen of precious metal being tested, typically gold, produces a galvanic reaction that allows the selection of a calibration chart from those stored in the memory of the meter for subsequent tests of that precious metal.

As is seen in the test chart below, each test using platinum or titanium wire, respectively, in the pen probes 11 were calibrated using 18K gold specimens. Thus, the readings were zeroed out at a comparison zero millivolts. The test readings for each test specimen, with the 18K specimen being the calibration setting, and the other reading numbers are the differential millivolt readings (delta) from the 18K specimen. As an example, the platinum wire pen probe 11 obtained a 17 millivolt differential reading for the 22K specimen, while the pen probe 11 with the titanium wire obtained a 35 mv differential reading. This slightly more than doubling of the differential reading of titanium over platinum provides a much higher confidence in the identification of an unknown test sample.

Test Results—Platinum Wire vs. Titanium Wire

All tests use a 3.3 FD source of current, a 5 k/10 k differential ladder, and 40 mm of wire:

| | Platinum | Titanium | Differential | Delta |
|---|---|---|---|---|
| Gold Karats | Wire | Wire | Reading | Improvement |
| 24K | 40 mv | 50 mv | 10 mv | 25% |
| 22K | 17 mv | 35 mv | 18 mv | 105% |
| 18K | 0 | 0 | 0 | |
| 14K | −116 mv | −125 mv | −9 mv | 7% |
| 10K | −306 mv | −250 mv | 56 mv | 18% |
| 8K | −356 mv | −480 mv | −124 mv | 34% |

Other testing has obtained similar unexpected results. The pen probe 11 having titanium wire to create the galvanic reaction consistently provided a greater millivolt differential, particularly in the higher Karat values of the gold specimen. With the understanding that improvement in the millivolt differential readings for the higher grades of gold are of great desire, the switching from platinum wire to titanium wire provided that substantial improvement, i.e., 25 to 105 percent improvement in the determination of gold grades over 18K. From a manufacturing standpoint, the cost of platinum per kilogram is presently about $32,000, while the price of titanium is about $100 per kilogram, which is a highly favorable price differential for titanium wire. It is also to the best of Applicant's knowledge that all gold testing devices utilizing a pen probe creating a galvanic reaction utilize platinum or platinum coated wires in the pen probe 11.

Increased Delta in Gold Testing Response Compared to Platinum and Platinum Coated Wire

The use of titanium wire in the pen probe significantly increases the effectiveness at both ends of the gold spectrum. Both 22 k and 24 k gold are the most difficult to read accurately and the improved delta from the use of a titanium wire makes a major difference. The galvanic response to different gold Karat (from 18 k) are plotted above in the calibration table. The response curves for platinum coated wire or pure platinum wire vs titanium wire are considerably different. One measurement is to consider the number of millivolts of delta per Karat. For example, on pure platinum wire, using the 18K standard as the calibration zero point, a 22K test specimen may read a delta of 17 mv, which computes to a 4.25 mv per Karat for the four Karat rise in quality.

On the other hand, using titanium wire to test the same gold specimen, the 22K test specimen will read a 35 mv delta instead of 17 mv as found with the platinum or platinum coated wire pen probe. Calculating the millivolts per Karat, 35 mv divided by 4K quality differential yields a per Karat delta of 8.75 mv, which is a significant increase, and important because 4.25 millivolts can easily be eaten up in pen stability, electrical noise and other environmental factors. Therefore, the conventional precious metal test apparatus 10 lacks sufficient effectiveness to have confidence in the identification of a 22K or 24K test specimen, when using a platinum wire pen probe. At a delta of 8.85 mv/K, the identification of a 22K test specimen will have much more certainty.

Increased Pen Stability and Speed of Readings

The use of a titanium wire pen probe has seen significant gains in both stability and speed, especially at the lower end of the gold spectrum (i.e., 14K, 10K and 8K). Readings that used to take several seconds with the conventional platinum wire pen probe now take less than one second to acquire. Similar, though smaller, gains have been obtained with the upper end of the gold spectrum (22K and 24K), as well. Overall, the pen is much more stable in all aspects, as environmental variables, such as humidity and contamination, have less impact on the readings when a larger delta is used to ascertain the quality of the test specimen.

Manufacturing Advantages

When the pen probe 11 is touched to a piece of 18K gold calibration specimen to start the calibration process, the meter 20 will show a reading in millivolts which will likely vary from one pen probe to another. This variance from one pen probe to another is called a reading “offset”. As part of the calibration process, this reading can be considered simply as a starting point, but the reading also varies from environmental factors, such as temperature and humidity, among other factors. Touching the pen probe 11 to another sample of gold that has a different quality, measured in Karats, results in a second reading in millivolts. The difference between the second millivolt reading and the initial millivolt reading on the 18K gold calibration specimen provides the delta as described above.

For example, an initial 18K gold calibration reading of 800 mv, compared to a second reading taken on a 22K test specimen that gives a reading of 835 mv, a delta of 35 mv can be computed by subtracting the 18 k reading from the 22 k reading. It doesn't matter what the 18 k offset is, we just need to know what it is so that we can subtract it from all future readings. The calibration curve shape has not changed, only the offset, which typically varies from pen probe to pen probe and even drifts during a single usage session. However, it should be noted by one skilled in the art that an actual slight change in the curve shape can occur, depending on the pen probe's offset. A pen probe 11 offsetting at 600 mv will have a slightly different curve shape than a pen offsetting at 900 mv. The shape of the curve mostly changes at the extreme ends of the gold spectrum (i.e., 8K vs 24K). The change in shape is small and usually only requires a minor adjustment to the calibration curve. Lower offset pens will warp the curve a little more than higher offset pens will and in different directions.

An algorithm can be used to compute this correction. Such a correction is usually called a “matrix correction” and is descriptive of the algorithm that creates the correction. From the testing of hundreds of pen probes of many different offsets, empirical data was acquired to generate a single point correction at each end of the calibration table (i.e., 8K and 24K) at many offsets, which is applied to the calibration table at those points. The rest of the calibration table is adjusted by performing a linear extrapolation to fill in the rest of the table.

The repeatability in offset readings titanium wire pen probes are far better than with platinum wire pen probes. This repeatability of offset readings, as a function of the measured offset from one pen probe to another in the manufacturing process, is an important manufacturing advantage gained with the titanium wire pen probes. For example, the variation in offset readings from one platinum wire pen probe to another can vary wildly, which can vary over a range of more than 150 mv, results is a large number of rejections of platinum wire pen probes, and a resultant increase in the cost of manufacturing. The variation from one titanium wire pen probe to another is typically in the range of only about 10 mv to 20 mv. This drastic reduction in offset variations in the manufacture of pen probes results in a near zero product rejection during manufacturing and testing of the finished pen probes. The end result is a significantly lower manufacturing cost in titanium pen probes, not only with respect to the cost of materials, but also with respect to the stability of the titanium wire pen probes.

To round out the disclosure of the testing apparatus 10, one skilled in the art would note that the testing apparatus 10 further includes a meter 20 including a housing 21 in which is mounted a printed circuit board 22 including a port 23 to which the jack 15 can be detachably connected. Also electrically connected to the circuitry 22 is a test pad 25 that is formed from substantially any electrically conductive material, including copper or aluminum.

The meter 20 is also constructed with an indicator device, preferably constructed with a light-emitting diode (LED) indicator bar 24 that reflects the results of the measurement of the galvanic current, as will be discussed in greater detail below. In addition, the housing 21 supports a calibration switch 26 that is operable to initiate the calibration procedure, as will be described in greater detail below. The housing 21 also supports an indicator, such as four LEDs, that reflects the status of the operation of the testing apparatus 10, including a first LED 27 to indicate the power is turned on and the testing apparatus 10 is ready, a second LED 28 to indicate that the calibration procedure is finished, a third LED 29 to indicate that the battery is low on power and needs to be replaced, and a fourth LED 29a to indicate that the pen probe 11 needs to be replaced. The meter 20 has a three position on/off switch 30 that is movable to an off position, an external power position, and a battery power position. The housing 21 also preferably supports a port 31 for connection to a source of external power, such as 110 VAC electrical current through the use of an adapter (not shown).

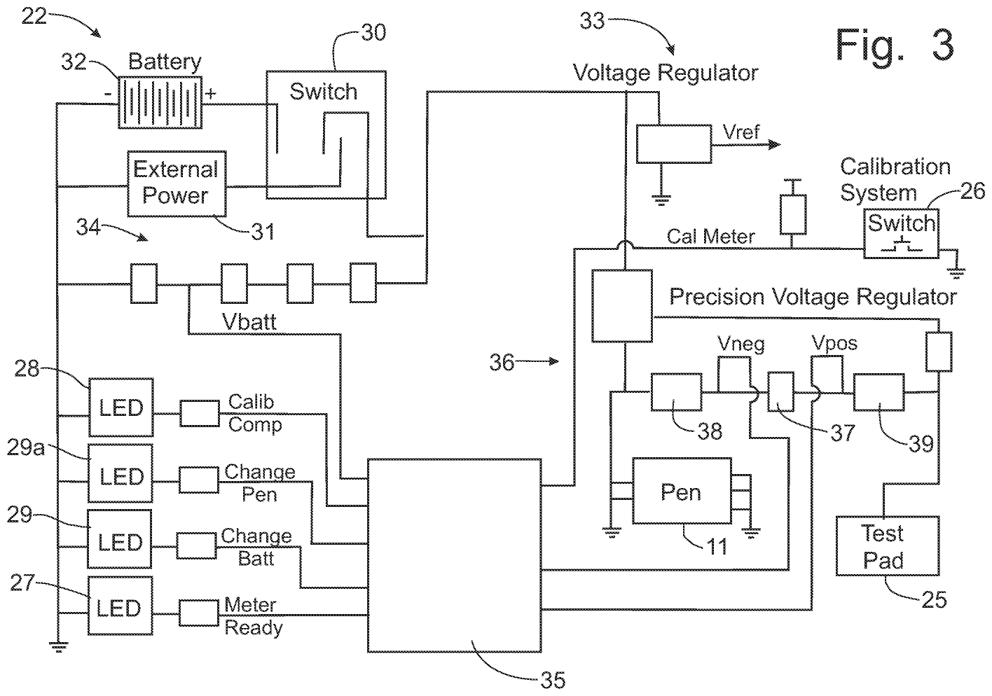

The circuitry 22 is reflected in the schematic diagram of FIG. 3. Either a battery 32 or a source of external electrical power connected through the port 31 provides an electrical current into the circuitry 22 that is regulated to a base reference voltage (Vref) by the voltage regulator 33. The microprocessor 35 monitors the voltage divider circuit 34 to ensure sufficient operational voltage (Vbatt) to run the testing apparatus 10. When operational voltage drops below a minimum requirement, the microprocessor 35 illuminates the third LED 29 to indicate a battery change is needed. Once the microprocessor 35 confirms that sufficient operational voltage is available and that the voltage (Vref) is properly regulated, the first LED 27 is illuminated to indicate that the testing apparatus 10 is ready to start operation.

Once calibrated, as will be described in greater detail below, the testing apparatus 10 is able to determine the quality (i.e., precious metal content) of an object. The object to be tested is placed onto the test pad 25 and the pen probe 11 is moved into engagement with the object by touching the exposed fiber tip 16 on the object, completing the electrical circuit within the meter 20. A galvanic reaction then occurs within the pen probe 11, creating a weak voltage within a range of approximately 60 millivolts, which is detected by the microprocessor 35 monitoring the voltage (Vneg and Vpos) in the metering circuit 36 on opposite sides of the resistor 37. The galvanic voltage generated in the pen probe 11 by completing the electrical circuit in the meter 20 through the object being tested is directly proportional to the precious metal content within the object and opposes the forward biased diodes 38, 39 in the metering circuit 36. The opposition to this forward bias decreases the voltage across the resistor 37. Thus, the galvanic voltage (Vdiff) is the difference between the voltage (Vneg and Vpos) on opposite sides of the resistor 37.

The microprocessor determines this galvanic voltage (Vdiff) and compares the galvanic voltage with known galvanic responses stored in a look-up table within the microprocessor 35, Since the galvanic reaction (Vdiff) is directly proportionate to the percentage of precious metal within the object being tested, the microprocessor can determine the percentage from the values on the look-up table and illuminate the LEDs in the indicator bar corresponding to the percentage of precious metal detected in the measurement. Thus, the indicator bar will provide an indication of the Karat weight of the gold in the object, but can also determine the percentage of silver and platinum within the object if the object is not made of gold.

The microprocessor 35 incorporates a system for automatically calibrating the subtle differences in the testing apparatus 10, including manufacturing irregularities within the pen probe 11 that normally occur, as well as differences relating to the operating environment, such as changes in the temperature and humidity, and the longevity of use of the pen probe 11. The calibration system has two primary components, look-up tables stored within the electronic microprocessor 35 and a recalibration procedure operated by the electronic microprocessor 35. The look-up tables store the galvanic strengths of gold, silver and platinum that have been normalized for a new pen probe 11 having a full sacrificial metal wire 18 and a full reservoir 17 of electrolyte, under the operating environmental conditions of a temperature at 25° C. and 40% humidity. The calibration procedure realigns the testing apparatus 10 to known operating parameters based upon a current measurement of a known parameter.

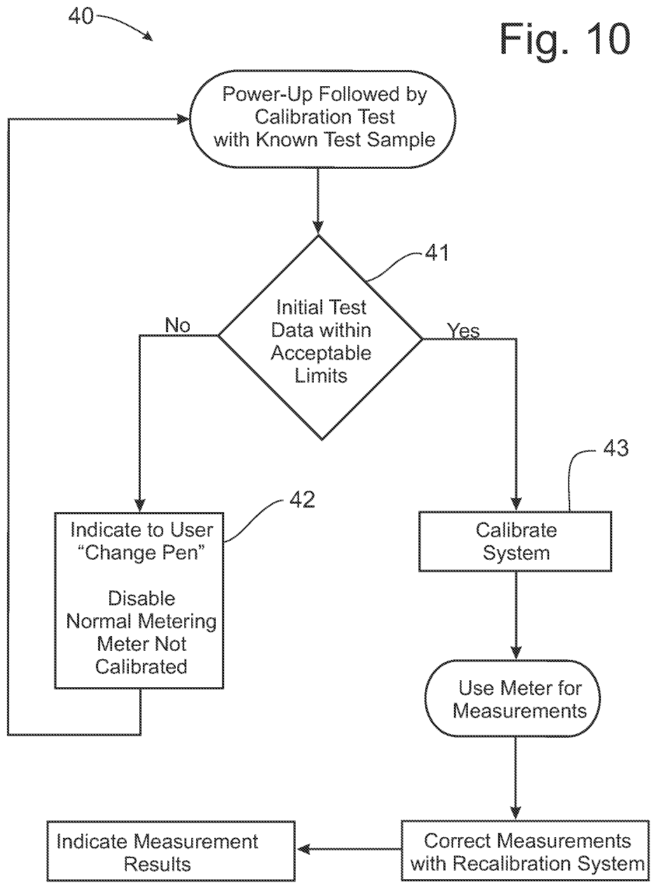

As is reflected in FIG. 10, the testing apparatus 10 must be calibrated at initial power-up to establish the base operating reference point for the recalibration system 40. The calibration system 40 starts with the placement of a known test specimen (not shown), having a known specific percentage of precious metal, on the test pad 25 and touches the test specimen with the pen probe 11. The first test is at step 41 to determine if the initial reading of the galvanic reaction corresponding to the known specimen is within normal acceptable limits. If not, the microprocessor illuminates the fourth LED 29a at step 42 to provide an indication that the pen probe 11 needs to be replaced. Assuming that the initial reading is acceptable, the operator then activates the calibration system 40 at step 43 by depressing the momentary switch 26 to initiate the calibration procedure.

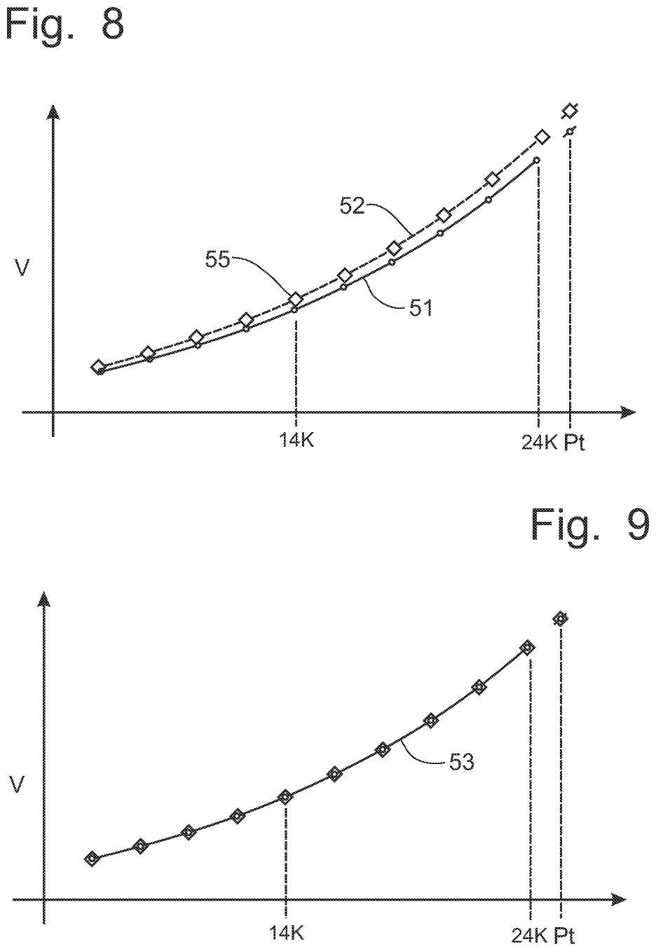

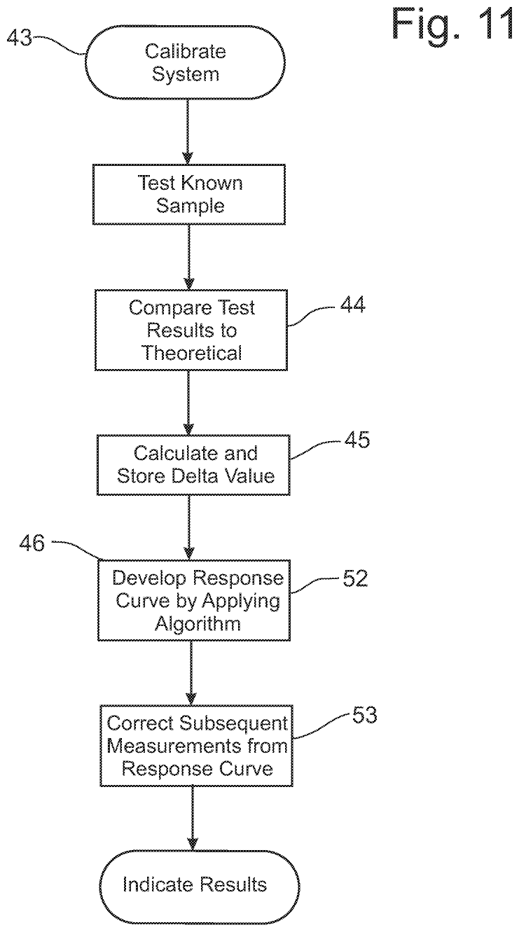

As depicted in FIG. 11, the microprocessor 35 compares at step 44 the initial reading of the galvanic reaction of the known specimen to the theoretical value shown for the known test specimen within the look-up table within the microprocessor 35. Assuming that there is a difference between the initial reading and the corresponding theoretical value from the look-up table, the delta value is calculated and stored in the microprocessor 35 at step 45, as shown in FIG. 7 as data point 55. The microprocessor 35 then develops at step 46 a response curve 52 using the algorithm corresponding to the curve 51 of the theoretical values from the look-up table, as is represented in FIGS. 6 and 8. This response curve 52 is then recalibrated, as is represented in FIG. 9, by the differential between the theoretical value curve 51 and the response curve 52 to develop a recalibration curve 53 that is used to correct all subsequent readings of objects being tested, until the next calibration procedure is undertaken.

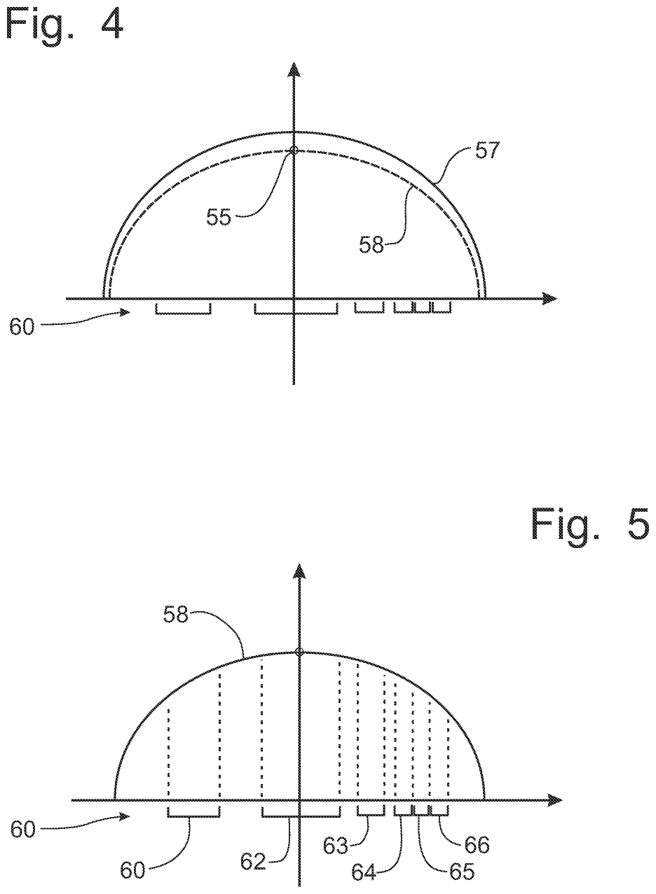

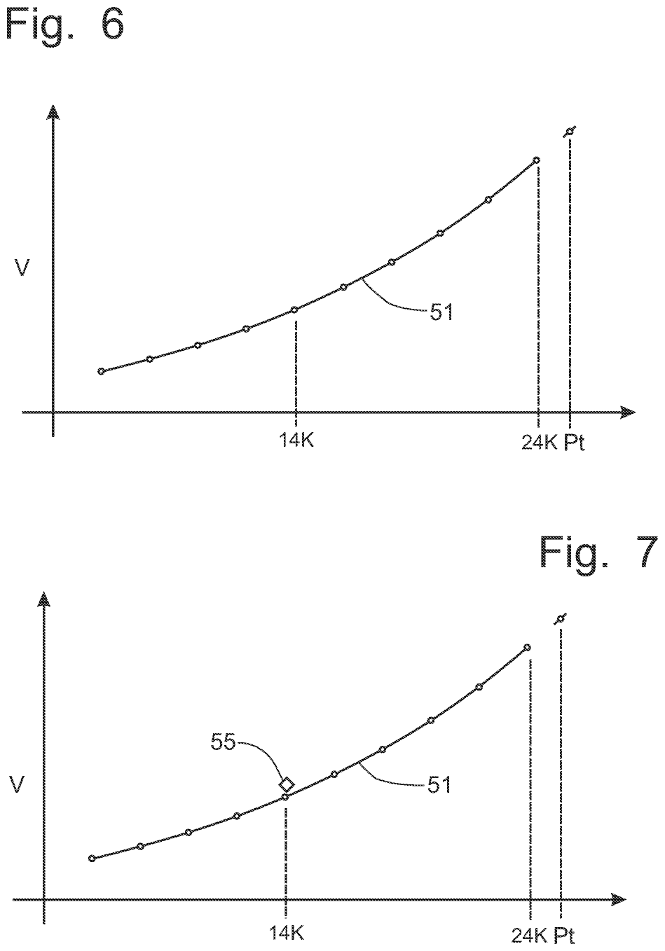

The end result of the recalibration procedure is depicted in FIGS. 4 and 5. The theoretical galvanic voltages within the look-up tables can be plotted on a graph somewhat as a parabolic curve 57. The initial reading 55 can be above or below the corresponding theoretical reading along the Y-axis. When plotted, the response curve 52 results in a calibrated parabolic curve 58 which is then used to signify the Karat weight of the object being subsequently tested by reference to Karat bands 60 with 10K gold falling within the band 61, 14K gold falling into band 62, 18K gold falling in band 63, 22 K gold falling into band 64, 24K gold falling into band 6556, and platinum falling into band 66. The Karat bands 60 correspond to the LED brackets depicted on the indicator bar 24 on the meter 20.

Because of the changes in the variables involved in the testing of precious metal with the testing apparatus 10, the calibration procedure should be run each time the meter 20 is powered up. Furthermore, the ion exchange in the electrolyte can deteriorate the sensitivity of the testing apparatus 10; therefore, the calibration procedure should also be run after a significant number of objects have been tested even if the meter has not been powered down. Likewise, moving the meter 20 from one environmental situation into a significantly different environmental situation would preferably incur an invoking of the calibration procedure. The calibration of the meter 20 only takes a few seconds and should not be an impediment to implement when accuracy is important to the operator. This process of measuring the precious metal content of an object is completed; start to finish, in just a few hundredths of a second. Thus, an extremely efficient method is provided to characterize samples containing precious metals.

With the increased galvanic charge generated through the use of the titanium wire 18 within the test pen probe 11, the testing apparatus 10 can be utilized to distinguish certain grades of non-precious metals. One example would be to distinguish the 304 grade of stainless steel from the 316 grade of stainless steel. The difference between the two grades of stainless steel is about 4% chromium. This chromium distinction makes the value of the two stainless steel grades significantly different. Presently, the recycling industry utilizes expensive XRF guns to determine the difference between the two grades of stainless steel. The titanium wire test pen probe 11 can distinguish the two grades of stainless steel and other non-precious metals at a small percentage of the cost of an XRF gun.

It will be understood that changes in the details, materials, steps and arrangements of parts which have been described and illustrated to explain the nature of the invention will occur to and may be made by those skilled in the art upon a reading of this disclosure within the principles and scope of the invention. The foregoing description illustrates the preferred embodiment of the invention; however, concepts, as based upon the description, may be employed in other embodiments without departing from the scope of the invention.

1. A testing apparatus for determining a quality measurement of a metal within a specimen being tested, comprising:
a meter including:
a microprocessor having look-up tables stored therein;
an indicator operably coupled to said microprocessor;
a source of electrical power connected to said microprocessor;
a test pad formed from electrically conductive material; and
an electronic circuit interconnecting said microprocessor, said indicator bar, said source of electrical power and said test pad;
a probe connected to said meter and including:
a casing;
a reservoir mounted within said casing and containing a supply of electrolyte;
a fiber tip supported in said casing and having a first end coupled to said reservoir to receive electrolyte therefrom and a second end exposed from said casing;
an electrical connector interconnecting said pen probe and said metering device; and
a galvanic reaction apparatus mounted within said reservoir and having a non-precious metal wire connected to said electrical connector, said galvanic reaction apparatus generating a galvanic voltage when said fiber tip completes an electrical circuit.

2. The testing apparatus of claim 1 further comprising:
a calibration system operably associated with said microprocessor to operate a calibration procedure that utilizes a test reading from a known test specimen and compares said test reading with a theoretical test reading stored in said look-up tables.

3. The testing apparatus of claim 2 wherein said calibration procedure applies an algorithm from the theoretical test readings for different grades of metal being tested to develop a response curve.

4. The testing apparatus of claim 3 wherein said theoretical test readings apply to the Karat weights of gold in said look-up tables.

5. The testing apparatus of claim 3 wherein said theoretical test readings apply to grades of non-precious metals.

6. The test apparatus of claim 5 wherein said non-precious metal is stainless steel.

7. The testing apparatus of claim 3 wherein said source of electrical power is one of a battery and an external source of electrical power coupled to said electronic circuit by an external port.

8. The testing apparatus of claim 7 wherein said indicator comprises an array of light-emitting diodes that said microprocessor illuminates in response to the test reading from an object interconnecting said test pad and said fiber tip of said probe.

10. The testing apparatus of claim 1 wherein said non-precious metal wire is formed from titanium.

11. A pen probe for creating a galvanic reaction in a gold testing apparatus having a meter connected to said pen probe, said meter having a test pad formed from an electrically conductive material, comprising:
electrolyte;
a casing;
a reservoir mounted within said casing and containing a supply of a fiber tip supported in said casing and having a first end coupled to said reservoir to receive electrolyte therefrom and a second end exposed from said casing;
an electrical connector interconnecting said pen probe and said metering device; and
a galvanic reaction apparatus mounted within said reservoir and having a non-precious metal wire connected to said electrical connector and extending into said reservoir of electrolyte, said galvanic reaction apparatus generating a galvanic voltage when said fiber tip completes an electrical circuit with said test pad.

12. The pen probe of claim 11 wherein said non-precious metal wire is made from titanium.

13. In a pen probe for creating a galvanic reaction in a metal testing apparatus having a meter connected to said pen probe, said pen probe including a casing, a reservoir mounted within said casing and containing a supply of electrolyte, a fiber tip supported in said casing and having a first end coupled to said reservoir to receive electrolyte therefrom and a second end exposed from said casing, an electrical connector interconnecting said pen probe and said metering device, and a galvanic reaction apparatus mounted within said reservoir, an improved galvanic reaction apparatus comprising:
a titanium wire connected to said fiber tip and to said electrical connector, said titanium wire generating a galvanic voltage when said fiber tip completes an electrical circuit with said test pad.

14. The pen probe of claim 13 wherein said pen probe is operably connected to a meter to process the generated galvanic voltage.

15. The pen probe of claim 14 wherein said meter includes:
a microprocessor having look-up tables stored therein;
an indicator operably coupled to said microprocessor;
a source of electrical power connected to said microprocessor;
a test pad formed from electrically conductive material; and
an electronic circuit interconnecting said microprocessor, said indicator, said source of electrical power and said test pad.

16. The pen probe of claim 15 wherein said meter operates a calibration system operably associated with said microprocessor to provide a calibration procedure that utilizes a test reading from a known test specimen and compares said test reading with a theoretical test reading stored in look-up tables stored within said microprocessor, said calibration procedure applying an algorithm from the theoretical test readings for different grades of metal being tested to develop a response curve.

17. The pen probe of claim 16 wherein said pen probe and said associated meter can be used to distinguish grades of non-precious metals.

18. The pen probe of claim 17 wherein said non-precious metals includes stainless steel.

19. The pen probe of claim 16 wherein said pen probe and said associated meter can be used to distinguish quality levels of precious metals.

20. The pen probe of claim 19 wherein said quality levels of precious metals includes the Karat level of gold.
